(19) United States
(12) Patent Application Publication
(10) Pub. No.: US 2026/0271851 A1
(43) Pub. Date: Sep. 17, 2026

(54) A SPINACH PLANT RESISTANT TO DOWNY MILDEW AND A RESISTANCE GENE

(71) Applicant: Enza Zaden Beheer B.V., Enkhuizen (NL)

(72) Inventors: Faira Suidgeest, Alkmaar (NL); Charikleia Schoina, Hoorn (NL); Jan Ane Dijkstra, Enkhuizen (NL); Denise Blom, Bovenkarspel (NL); Trinette Van Selling, Enkhuizen (NL)

(21) Appl. No.: 19/128,361

(22) PCT Filed: Feb. 22, 2023
(86) PCT No.: PCT/EP2023/054392

§ 371 Date: May 8, 2025

(30) Foreign Application Priority Data
Nov. 11, 2022 (NL) 2033517

(51) Int. Cl. A01H 6/02; A01H 5/12

(52) U.S. Cl. CPC A01H 6/028; A01H 5/12

(57) ABSTRACT

The present invention relates to a spinach plant that is resistant to downy mildew caused by Peronospora farinosa. The present invention further relates to a resistance gene that confers resistance to downy mildew in spinach plants and methods for obtaining a spinach plant that is resistant to downy mildew.

The present invention relates to a spinach plant that is resistant to downy mildew caused by Peronospora farinosa. The present invention further relates to a resistance gene that confers resistance to downy mildew in spinach plants and methods for obtaining a spinach plant that is resistant to downy mildew.

Spinach (Spinacia oleracea) is an open field crop grown in many diverse environments. Spinach is a diploid crop that grows well in areas that have a cool, wet spring period, cool summers and dry autumns. Optimal soil conditions include well-drained soils and a pH above 6. Nowadays, spinach breeding mainly focusses on disease resistance (e.g. downy mildew), crop yield and improved nutritional value.

Breeding and screening activities helps to select varieties in the main production regions, where local market adaptation and dynamic resistance are important factors to success. Spinach breeding programmes are developed that aim to provide for varieties for all market segments; the fresh (babyleaf) market, bunching market as well as frozen and canned products. Several specific varieties of spinach are available on the market, within the main types: smooth, savoyed and oriental types. The spinach market is growing rapidly worldwide and much of research is being performed to improve genetics of the spinach for instance in relation to disease resistance and reducing the need for biochemicals or pesticides, and to improve both crop yield and crop quality. Another goal of the breeding programs is to provide for spinach varieties with broad resistance patterns to downy mildew caused by Peronospora farinosa (also known more recently as Peronospora effuse), and ideally already taking future strains into account.

Downy mildew refers to several types of oomycete microbes that are parasites of plants. Downy mildew can originate from various species, but mainly of Peronospora, Plasmopara and Bremia. Downy mildew is a problem in many food crops, and in spinach caused by Peronospora farinosa sp. (Pfs), affecting the production of this crop worldwide. Downy mildew is one of the most problematic diseases in spinach. Spinach Downy mildew infection show symptoms of discoloured areas and irregular yellow patches on upper leaf surfaces in combination with white, grey or purple mould located on the other side of the leaf surface below. Lesions may eventually dry out and turn brown. Disease is spread from plant to plant by airborne spores.

Fungicides can be used to control Peronospora farinosa, but eventually Peronospora farinosa becomes immune to these chemicals, because over time the pathogen also acquires resistance to fungicides. In addition, the market wishes to reduce the use of such chemicals in the production of food crops. Therefore, it is of the utmost importance to find other methods to control Peronospora farinosa infection. Most preferably is to identify a resistance gene that gives broad resistance against Peronospora farinosa. Also one or more resistance genes can be combined to achieve a broad scope and durable resistance against Peronospora farinosa. Therefore, identification of new resistance genes is a promising alternative.

Nineteen official races of Peronospora farinosa have been identified to date (Pfs1 to Pfs19) and characterization is based on qualitative disease reactions on a set of host differentials, an approach widely used to identify races of many plant pathogens. For spinach, the current set of differentials is comprised of new and old commercial hybrids as well as open-pollinated cultivars and breeding lines. In addition, the pathogen under pressure mutates to break down the disease resistance and new disease resistance in crops is needed to control infection. Especially in spinach the occurrence of resistant downy mildew is particularly complex as there are many different races, and new resistant downy mildew species, i.e. races that break current resistances emerge all the time. Breakthrough can occur as quick as within 4 to 6 months. The main problem is that the present spinach varieties on the market combining different resistances become very fast outdated as Peronospora farinosa quickly evolves new virulent races. With new races of downy mildew popping up in spinach over the last several years, it becomes increasingly more difficult to stay a step ahead of the devastating disease.

Newly developed hybrids by combining different resistances become very fast outdated as Pfs quickly evolves new virulent races. The search of new resistance genes and their implementation in spinach breeding is a great challenge. Most known resistances are deriving from single dominant genes (NBS-LRR) and at present there is no single resistance gene available that provides full spectrum resistance to all races/fysio's of Peronospora farinosa. Therefore, it is an advantage to combine or stack multiple resistance genes into a spinach plant, such that a plant is obtained that comprises multiple resistance genes and is resistant to all Peronospora farinosa fysios, or at least is resistant to as many Peronospora farinosa types as possible.

Considering the above, there is a need in the art to develop a more diverse and durable resistance in spinach and to provide spinach plants that are resistant to downy mildew caused by Peronospora farinosa and wherein these plants have a broad-spectrum resistance against this pathogen that causes downy mildew. Furthermore, it is an object of present invention to provide a method to obtain such downy mildew resistant plants. There is a need for more diversity of alleles, so that more genetic variation can be achieved in commercial hybrids, making it harder for pathogens such as Peronospora farinosa to adapt. The broader the resistance of these alleles, the more effectively they can be used in the development of resistant plants.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a spinach plant that is resistant to downy mildew caused by Peronospora farinosa (Pfs), wherein said one or more resistance genes encode for a protein comprising an amino acid domain having at least 98%, preferably at least 99/a, more preferably 100% sequence identity with SEQ ID No.2. The identification of novel candidate dominant Pfs resistance genes of present invention are also known as CC-NBS-LRR genes. A novel resistance gene were found, more specifically 1084/1096-T39 obtained by sequencing of locus 1 and gene mapping of Peronospora farinosa resistance genes in Spinach. This resistance gene encodes for a protein that comprises the specific amino acid domain of SEQ ID No.2. This protein domain comprises three specific LRR (leucine rich repeat) domains. The primary function of these LRR domains appear to be to provide a versatile structural framework for the formation of protein-protein interactions in relation the disease resistance in the plant. It is toughed that those domains determine effector recognition and therefore disease susceptibility/resistance. The majority of disease resistance genes in plants encode nucleotide-binding site leucine-rich repeat proteins, also known as NBS-LRR proteins (encoded by R genes). These proteins are characterized by nucleotide-binding site (NBS) and leucine-rich repeat (LRR) domains as well as variable amino- and carboxy-terminal domains and are involved in the detection of diverse pathogens, including bacteria, viruses, fungi, nematodes, insects and oomycetes. There are two major subfamilies of plant NBS-LRR proteins defined by the Toll/interleukin-1 receptor (TIR) or the coiled-coil (CC) motifs in the amino-terminal domain and are both involved in pathogen recognition. Most known resistances in spinach were identified from a single dominant gene (NBS-LRR), called Locus 1 located, which is highly variable. Although many alleles have been identified in many different wild spinach accessions, it is still unknown whether they are functional or not, e.g. provide Pfs resistance.

In spinach these novel resistance genes were mapped on locus 1 on chromosome 3 in the spinach genome (spinachbase.org). Like many other NBS-LRR genes, locus 1 is highly variable and more than 600 alleles have been identified. The coding sequences of 1084/1096-T39 is represented by SEQ ID No.3 (partial sequence), more specifically SEQ ID No. 7.

According to a preferred embodiment, the present invention relates to the spinach plant wherein said one or more resistance genes encode for a protein, wherein said protein comprises an amino acid sequence having at least 96%, preferably at least 98%, more preferably 100% sequence identity with SEQ ID No.4, or with SEQ ID No.8, preferably SEQ ID No.8.

According to a preferred embodiment, the present invention relates to the spinach plant wherein said one or more resistance genes comprise a coding sequence having at least 96%, preferably at least 98%, more preferably 100% sequence identity with SEQ ID No.1, SEQ ID No.3, or SEQ ID No.7, preferably SEQ ID No.7.

According to another preferred embodiment, the present invention relates to the spinach plant wherein said plant is at least resistant to Peronospora farinosa races Pfs1 to Pfs19.

According to another preferred embodiment, the present invention relates to the spinach plant wherein said one or more resistance genes is derived from deposit number NCIMB 44064. Seeds of Spinacia oleracea plant according to present inventions were deposited on 24 Oct. 2022 at NCIMB Ltd, Ferguson Building, Craibstone Estate Bucksburn, AB21 9YA Aberdeen, United Kingdom.

The present invention, according to a second aspect, relates to seed produced by a spinach plant according to present invention.

The present invention, according to a third aspect, relates to a resistance gene that confers resistance to downy mildew in spinach plants, wherein the gene encodes for a protein comprising an amino acid domain having at least 98%, preferably at least 99/6, more preferably 100% sequence identity with SEQ ID No.2.

According to yet another preferred embodiment, the present invention relates to the resistance gene, wherein said protein comprises an amino acid sequence having at least 96%, preferably at least 98%, more preferably 100% sequence identity with SEQ ID No.4 (partial sequence), or with SEQ ID No.8, preferably SEQ ID No.8. The novel resistance gene encode for proteins that confer broad Pfs resistance in spinach.

According to a preferred embodiment, the present invention relates to the resistance gene, wherein said resistance gene comprises a coding sequence having at least 96%, preferably at least 98%, more preferably 100% sequence identity with SEQ ID No.1, or SEQ ID No.3, or SEQ ID No.7, preferably SEQ ID No.7. The resistance gene comprises a coding sequence of SEQ ID No. 1 which encodes for a specific protein domain that comprises the specific amino acid domain comprised of three specific LRR domains for the formation of protein-protein interactions in relation the disease resistance in the plant, and determine effector recognition and therefore disease susceptibility/resistance. SEQ ID No.7 comprises the full coding sequence of the resistance gene.

According to yet another preferred embodiment, the present invention relates to the resistance gene, wherein the gene provides resistance to Peronospora farinosa races Pfs1 to Pfs19 in spinach.

According to a further aspect, the present invention relates to a method for providing a spinach plant that is resistant to downy mildew, wherein the method comprises the steps of introducing or modifying the one or more resistance genes as described above into the genome of the spinach plant.

According to another preferred embodiment, the present invention relates to the method, wherein the introduction or modification of the one or more Pfs resistance genes is achieved by genome editing techniques, CRISPR Cas, or mutagenisis techniques.

The present invention, according to a further aspect, relates to a method for providing a spinach plant that is resistant to downy mildew, wherein the method comprises the steps of
a) providing a spinach plant comprising one or more resistance gene(s) of the present invention,
b) crossing the spinach plant of step a) with a susceptible spinach plant,
c) optionally, selfing the plant obtained in step b) for at least one time,
d) selecting the plants that are resistant to downy mildew.

According to another preferred embodiment, the present invention relates to the method, wherein the spinach plant is resistant to downy mildew caused by Peronospora farinosa races Pfs1 to Pf19.

According to a preferred embodiment, the present invention relates to the method, wherein the one or more resistance gene(s) is obtained from deposit number NCIMB 44064.

The present invention, according to a further aspect, relates to a method for selecting a spinach plant that is resistant to downy mildew, wherein the method comprises the step of establishing in the genome of the plant or seed thereof, the presence of a resistance gene as disclosed herein, and/or establishing in the genome of the plant or seed thereof the presence of the resistance gene by using the SNP markers SEQ ID No. 5 and SEQ ID No. 6.

The present invention will be further detailed in the following examples and figures wherein:

### EXAMPLES

### Genmapping of a Novel Candidate Dominant Resistance Gene

The resistance genes were mapped using a Bulk Segregant Analysis (BSA) approach. The RNA of multiple resistance families (originating from the F3) were pooled and compared to a pool of RNA of susceptible families. All F3 families derived from the same F2 plant. SNP markers were developed in regions where an increase in number of SNPs was observed. The markers were validated on the F2 population. Once a region of interest (ROI) could be identified and flanked by markers, a fine mapping approach was started. See Table 1 for the specific marker sequences used for the identification of 1084/1096-T39, wherein de SNP is indicated in a bold, underlined and small letter.

The identification of a novel candidate dominant resistance gene, CC-NBS-LRR genes, more specifically 1084/1096-T39, is obtained by gene mapping of Peronospora farinosa resistance genes in Spinach (S. oleracea). In spinach these novel resistance genes were mapped on locus 1 on chromosome 3 in the spinach genome. Resistance genes on locus 1 were previously mapped and validated, as described in WO2020/239572. Flanking markers were used in a F2 population of ~3000 samples to identify plants that contain a recombination between the two markers. Those recombinant plants were phenotyped with several Peronospora farinosa strains and genotyped by markers in the ROI. By combining the genotype and phenotype results, the region of interest could be reduced to 1084/1096-T39 gene (SEQ ID No.7), wherein said gene comprises a coding sequence of SEQ ID No. 1 that encodes for a protein that comprises the specific amino acid domain of SEQ ID No.2, comprising three specific LRR (leucine rich repeat) domains involved in the disease resistance mechanism. SEQ ID No.7 represents the complete coding sequence of the resistance gene 1084/1096-T39, and SEQ ID No. 8 represents the full protein sequence. SEQ ID No.3: represents a partial coding sequence of 1084/1096-T39 gene.

In order to identify new alleles that confer better resistance spectra, primers have been developed that are located upstream and downstream of the resistance gene candidate. Using PCR amplification in combination with long read technology sequencing, 1084/1096-T39 was identified and validated using markers. This marker is located 0 cM from Locus 1, meaning that the chance that the marker will be separated from the gene due to recombination events during meiosis is less than 1%. Showing a 100% correlation between the marker and the phenotype in a population size of >100 plants is proving that this gene is conferring the resistance.

TABLE 1

| Marker | Sequence | SEQ ID No. |
|---|---|---|
| 1084/1096-T39_ | ATTTCCCGAAAGGCTGAACCACACACAAA | 5 |
| marker- | CACTCTCCTAAACCAGCAGRTCAGTACAA | |
| resistent | GAGATTTAAATCATCGGCCTCAATATGGA | |
| | TACCTCAYTGTTcAAACTTAAAGGTGAATG | |
| | GTTAAATCACTGAGATCAATTAGCACTTTC | |
| | TTTCAAGGAGATWCTCCATTGCGAACAAG | |
| | ACACTAGTCTGCATTGCAAAACCT | |
| 1084/1096-T39_ | ATTTCCCGAAAGGCTGAACCACACACAAA | 6 |
| marker- | CACTCTCCTAAACCAGCAGRTCAGTACAA | |
| susceptible | GAGATTTAAATCATCGGCCTCAATATGGA | |
| | TACCTCAYTGTTgAAACTTAAAGGTGAATG | |
| | GTTAAATCACTGAGATCAATTAGCACTTTC | |
| | TTTCAAGGAGATWCTCCATTGCGAACAAG | |
| | ACACTAGTCTGCATTGCAAAACCT | |

Disease Resistance Test (Leaf Disc Test) for Peronospora farinosa in Spinach 1084/1096-T39

Spinach plants of present invention containing the 1084/1096-T39 gene are tested for resistance to Peronospora farinosa. The plants must be, at least in the second leaf stage and not yet flowering. Leaves of spinach plants that comprise the 1084/1096-T39 gene, were put in trays with moistened paperboard. The infected seedlings are suspended in 20 mL water, filtered by cheesecloth and the flow-through is collected in a spray flask. One tray is spray-inoculated with this Peronospora farinosa suspension. Spray leafs with inoculum and make sure that all the discs are wet. The trays are covered with a glass plate and stored in a climate chamber at 15° C. (12 hours of light). Seven to fourteen days days post inoculation infection leaves are phenotypically scored by eye on the presence of Peronospora farinosa (Pfs).

Spinach plants that comprise the 1084/1096-T39 gene were tested for resistance phenotype for Pfs. The leaves are scored based on symptoms of sporulation on upper (adaxial) or underside (abaxial side) of the leaf disc and scored according to the following scale:
9=No sporulation.
7-8=A small amount of sporulation (max 10 conidiophores).
5-6=Some sporulation on the edge of the disc.
3-4=Some sporulation in a small area of the punch or many sporulation on a piece of only 2-3 mm close to the edge.
2=Reasonable sporulation.
1=Strong sporulation.

The infection was validated by inclusion of the susceptible and resistant controls (Viroflay, Resistoflay, Califlay, Clermont, Campania, Boeing and Lazio), wherein the susceptible control scores at least a score of 1, 2 or 3. A score of 9 showed to be fully resistant. Furthermore, the degree of sporulation is qualified by the amount of sporulation and not the discoloration of the disc.

TABLE 2
Peronospora farinosa (Pfs) resistance phenotype spinach plants.

| | | | | | | | 1084/1096- |
|---|---|---|---|---|---|---|---|
| Pfs-type | Viroflay | Resistoflay | Califlay | Clermont | Campania | Boeing | T39 |
| 1 | 1 | 9 | 9 | 9 | 9 | 9 | 9 |
| 2 | 1 | 9 | 1 | 9 | 1 | 9 | 9 |
| 3 | 1 | 1 | 9 | 9 | 9 | 9 | 9 |
| 4 | 1 | 1 | 1 | 9 | 9 | 9 | 9 |
| 5 | 1 | 1 | 9 | 1 | 9 | 9 | 9 |
| 6 | 1 | 1 | 1 | 1 | 1 | 9 | 9 |
| 7 | 1 | 1 | 1 | 1 | 9 | 9 | 9 |
| 8 | 1 | 1 | 9 | 1 | 1 | 1 | 9 |
| 9 | 1 | 1 | 9 | 1 | 1 | 9 | 9 |
| 10 | 1 | 1 | 1 | 1 | 1 | 1 | 9 |
| 11 | 1 | 1 | 9 | 1 | 9 | 9 | 9 |
| 12 | 1 | 1 | 9 | 1 | 1 | 1 | 9 |
| 13 | 1 | 1 | 1 | 1 | 9 | 9 | 9 |
| 14 | 1 | 1 | 9 | 1 | 1 | 1 | 9 |
| 15 | 1 | 1 | 1 | 9 | 9 | 9 | 9 |
| 16 | 1 | 1 | 9 | 1 | 9 | 9 | 9 |
| 17 | 1 | 1 | 1 | 1 | 1 | 1 | 9 |
| 18 | 1 | 1 | 1 | 1 | 9 | 9 | 9 |
| 19 | 1 | 1 | 1 | 1 | 1 | 1 | 9 |

Table 2 shows an overview of the disease test performed with the isolates of Peronospora farinosa Pfs1 to Pfs19 on Spinach varieties. Results show that spinach comprising the 1084/1096-T39 resistance gene is resistance to at least Peronospora farinosa races Pfs1 to Pfs19. The control lines show to be susceptible to at least multiple downy mildew isolates. Only the plant of present invention is resistant to all Pfs, including the recent type Pfs 18 and 19.

1. A spinach plant that is resistant to downy mildew caused by Peronospora farinosa (Pfs), wherein the spinach plant comprises one or more resistance genes, wherein said one or more resistance genes encode for a protein comprising an amino acid domain having at least 98%, preferably at least 99%, more preferably 100% sequence identity with SEQ ID No.2.

2. The spinach plant according to claim 1, wherein said protein comprises an amino acid sequence having at least 96%, preferably at least 98%, more preferably 100% sequence identity with SEQ ID No.4 or with SEQ ID No.8, preferably SEQ ID No.8.

3. The spinach plant according to claim 1, wherein said one or more resistance genes comprise a coding sequence having at least 96%, preferably at least 98%, more preferably 100% sequence identity with SEQ ID No.1, SEQ ID No.3, or SEQ ID No.7, preferably SEQ ID No.7.

4. The spinach plant according to claim 1, wherein said plant is at least resistant to Peronospora farinosa races Pfs1 to Pfs19.

5. The spinach plant according to claim 1, wherein said one or more resistance genes is derived from deposit number NCIMB 44064.

6. A seed produced by a spinach plant according to claim 1.

7. A resistance gene that confers resistance to downy mildew in spinach plants, wherein the gene encodes for a protein comprising an amino acid domain having at least 98%, preferably at least 99%, more preferably 100% sequence identity with SEQ ID No.2.

8. The resistance gene according to claim 7, wherein said protein comprises an amino acid sequence having at least 96%, preferably at least 98%, more preferably 100% sequence identity with SEQ ID No.4, or with SEQ ID No.8, preferably SEQ ID No.8.

9. The resistance gene according to claim 7, wherein said resistance gene comprises a coding sequence having at least 96%, preferably at least 98%, more preferably 100% sequence identity with SEQ ID No.1, SEQ ID No.3, or SEQ ID No.7, preferably SEQ ID No.7.

10. The resistance gene according to claim 7, wherein the gene provides resistance to Peronospora farinosa races Pfs1 to Pfs19 in spinach.

11. A method for providing a spinach plant that is resistant to downy mildew, wherein the method comprises the steps of introducing or modifying a resistance gene according to claim 7 into the genome of the spinach plant.

12. The method according to claim 11, wherein the introduction or modification of the one or more resistance genes is achieved by genome editing techniques, CRISPR Cas, or mutagenesis techniques.

13. A method for providing a spinach plant that is resistant to downy mildew, wherein the method comprises the steps of
a) providing a spinach plant comprising a resistance gene according to claim 7,
b) crossing the spinach plant of step a) with a susceptible spinach plant,
c) optionally, selfing the plant obtained in step b) for at least one time,
d) selecting the plants that are resistant to downy mildew.

14. The method according to claim 11, wherein the spinach plant is resistant to downy mildew caused by Peronospora farinosa races Pfs1 to Pfs19.

15. The method according to claim 11, wherein the one or more resistance gene(s) is obtained from deposit number NCIMB 44064.

16. A method for selecting a spinach plant that is resistant to downy mildew, wherein the method comprises the step of establishing in the genome of the plant or seed thereof, the presence of a resistance gene according to claim 7, and/or establishing in the genome of the plant or seed thereof the presence of the resistance gene by using the SNP markers SEQ ID No. 5 and SEQ ID No. 6.

17. The method according to claim 13, wherein the spinach plant is resistant to downy mildew caused by Peronospora farinosa races Pfs1 to Pfs19.

18. The method according to claim 13, wherein the one or more resistance gene(s) is obtained from deposit number NCIMB 44064.


SEQUENCE LISTING (8 sequences)

SEQ ID NO: 1 - DNA, 174 bp, Spinacia oleracea
  source 1..174: mol_type = other DNA
atatttgattataaaggtgtatttgttaaccctggagctgtgttggcaaccctagagccaccttcaaatatcaagaggttagaggtgtggcattacgatggtacaacaattccagtatgggaaagagcagagattaattgggcaatctccctctcacatcttacaataactgat

SEQ ID NO: 2 - protein, 58 aa, Spinacia oleracea
IFDYKGVFVNPGAVLATLEPPSNIKRLEVWHYDGTTIPVWERAEINWAISLSHLTITD

SEQ ID NO: 3 - DNA, 2913 bp, Spinacia oleracea
  source 1..2913: mol_type = other DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttcaattggaagagttgttaatgttgggtcaagggggaagcaaggttgtagtgaccgcacgttctgagaaaacagcaaatgtcatagggaaaagacatttatatacactggaatgtttgtcaccagattattcatggagcttatttgaaatgtcggcttttcagaaagggcaggagcaggaaaaccatcacgaactagttcgtattgggaaaaagattgttgaaaaatgttataacaatccacttgtgataaccgtggttggaagtcttctttatggagaggagataagtaagtggcaatcatttgaaaggagtgagttggccaaaattggcaatggggataacaagattttgtcgatattgaagctcagttactacaatcttgcaaactctttgaagagttgttttagttattgtgcagtgtttcccaaggatcatgaaataaagaaggagatgttgattgaactttggatggcacaaggatatgttgtgccgttggatggtggtcaaagtatagaagatgctgccgaggaacattttgtaattttattacggagatgtttctttcaagatgtagtgaaggatgtatacggtgatgttaattctgttaaaatccacgacttgatgcacgatgtcgcccaagaagtgggcagagaggaaatctgtatagtgaatgataatacaaagaagttggctgataaaatccgtcacgtgggttgtgttgtcgatagagatccagaaatagtctttttatgtagcaataagattcgttcgtatattggtggtaattgtgaaaaacgttgggtggatacactaatagacaactggatgtgtcttagggtgttggacttgtcaaggtcggatgttaaaaatttgcctaattcaataggtaaattgttgcacttgaggtatcttaacctgtctcataatggagatctgttgatactccctgatgcaattacaagactgcataatttgcagacactacttttacatcggtgcagtagtttaaaggagttgccaaaagatttttgcaaattggtcaaactgaggcacttggatttatcgggttgtgaggatttgattggtatgccattgggaatggataggctaactagtcttagagtactgccattctttgtggtgggtaggaaggaacaaagtgttgatgatgagctgaaagccctaaaaggcctcaccgagataaaaggcgacattcgtattagaatctgttccaattatagaatagttgaaggcatgaatgacacaggaggagctgggtatttgaagagcatgaaacatctcactggggttaatattatatttgattataaaggtgtatttgttaaccctggagctgtgttggcaaccctagagccaccttcaaatatcaagaggttagaggtgtggcattacgatggtacaacaattccagtatgggaaagagcagagattaattgggcaatctccctctcacatcttacaataactgattccaaggagggggagggtgaatgggaagttggggatgcatttcagaagtgtgtatcttctttgagaagcctcaccataatcggaaatcacggaataaataaagtgatgagactgtctggaagaacagggttggagcatttcactctgttggactcactcgaactttcaaagatagaagaccaggaagatgagggcgaagacaacatcatattctggaaatcctttcctcaaaaccttcgcagtttgagaattgaagactctgacaaaatgacaagtttgcccatggggatgcagtacttaacctccctccaaaccctctatctacaccattgttatgaattgaattcccttccagaatggataagcagcttatcatctcttcaatccctgttcatatacaattgtccagccctaaaatcactaccagaagcaatgcggaacctcacctcccttcagagccttgtgatacggcggtgtccagacctaattgaaagatgcgaagaacccaacggcgaggactatcccaaaattcgacacatctccagaatt

SEQ ID NO: 4 - protein, 971 aa, Spinacia oleracea
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFQLEELLMLGQGGSKVVVTARSEKTANVIGKRHLYTLECLSPDYSWSLFEMSAFQKGQEQENHHELVRIGKKIVEKCYNNPLVITVVGSLLYGEEISKWQSFERSELAKIGNGDNKILSILKLSYYNLANSLKSCFSYCAVFPKDHEIKKEMLIELWMAQGYVVPLDGGQSIEDAAEEHFVILLRRCFFQDVVKDVYGDVNSVKIHDLMHDVAQEVGREEICIVNDNTKKLADKIRHVGCVVDRDPEIVFLCSNKIRSYIGGNCEKRWVDTLIDNWMCLRVLDLSRSDVKNLPNSIGKLLHLRYLNLSHNGDLLILPDAITRLHNLQTLLLHRCSSLKELPKDFCKLVKLRHLDLSGCEDLIGMPLGMDRLTSLRVLPFFVVGRKEQSVDDELKALKGLTEIKGDIRIRICSNYRIVEGMNDTGGAGYLKSMKHLTGVNIIFDYKGVFVNPGAVLATLEPPSNIKRLEVWHYDGTTIPVWERAEINWAISLSHLTITDSKEGEGEWEVGDAFQKCVSSLRSLTIIGNHGINKVMRLSGRTGLEHFTLLDSLELSKIEDQEDEGEDNIIFWKSFPQNLRSLRIEDSDKMTSLPMGMQYLTSLQTLYLHHCYELNSLPEWISSLSSLQSLFIYNCPALKSLPEAMRNLTSLQSLVIRRCPDLIERCEEPNGEDYPKIRHISRI

SEQ ID NO: 5 - DNA, 200 bp, synthetic construct
  source 1..200: mol_type = other DNA
atttcccgaaaggctgaaccacacacaaacactctcctaaaccagcagrtcagtacaagagatttaaatcatcggcctcaatatggatacctcaytgttcaaacttaaaggtgaatggttaaatcactgagatcaattagcactttctttcaaggagatwctccattgcgaacaagacactagtctgcattgcaaaacct

SEQ ID NO: 6 - DNA, 200 bp, synthetic construct
  source 1..200: mol_type = other DNA
atttcccgaaaggctgaaccacacacaaacactctcctaaaccagcagrtcagtacaagagatttaaatcatcggcctcaatatggatacctcaytgttgaaacttaaaggtgaatggttaaatcactgagatcaattagcactttctttcaaggagatwctccattgcgaacaagacactagtctgcattgcaaaacct

SEQ ID NO: 7 - DNA, 2925 bp, Spinacia oleracea
  source 1..2925: mol_type = other DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttcaattggaagagttgttaatgttgggtcaagggggaagcaaggttgtagtgaccgcacgttctgagaaaacagcaaatgtcatagggaaaagacatttatatacactggaatgtttgtcaccagattattcatggagcttatttgaaatgtcggcttttcagaaagggcaggagcaggaaaaccatcacgaactagttcgtattgggaaaaagattgttgaaaaatgttataacaatccacttgtgataaccgtggttggaagtcttctttatggagaggagataagtaagtggcaatcatttgaaaggagtgagttggccaaaattggcaatggggataacaagattttgtcgatattgaagctcagttactacaatcttgcaaactctttgaagagttgttttagttattgtgcagtgtttcccaaggatcatgaaataaagaaggagatgttgattgaactttggatggcacaaggatatgttgtgccgttggatggtggtcaaagtatagaagatgctgccgaggaacattttgtaattttattacggagatgtttctttcaagatgtagtgaaggatgtatacggtgatgttaattctgttaaaatccacgacttgatgcacgatgtcgcccaagaagtgggcagagaggaaatctgtatagtgaatgataatacaaagaagttggctgataaaatccgtcacgtgggttgtgttgtcgatagagatccagaaatagtctttttatgtagcaataagattcgttcgtatattggtggtaattgtgaaaaacgttgggtggatacactaatagacaactggatgtgtcttagggtgttggacttgtcaaggtcggatgttaaaaatttgcctaattcaataggtaaattgttgcacttgaggtatcttaacctgtctcataatggagatctgttgatactccctgatgcaattacaagactgcataatttgcagacactacttttacatcggtgcagtagtttaaaggagttgccaaaagatttttgcaaattggtcaaactgaggcacttggatttatcgggttgtgaggatttgattggtatgccattgggaatggataggctaactagtcttagagtactgccattctttgtggtgggtaggaaggaacaaagtgttgatgatgagctgaaagccctaaaaggcctcaccgagataaaaggcgacattcgtattagaatctgttccaattatagaatagttgaaggcatgaatgacacaggaggagctgggtatttgaagagcatgaaacatctcactggggttaatattatatttgattataaaggtgtatttgttaaccctggagctgtgttggcaaccctagagccaccttcaaatatcaagaggttagaggtgtggcattacgatggtacaacaattccagtatgggaaagagcagagattaattgggcaatctccctctcacatcttacaataactgattccaaggagggggagggtgaatgggaagttggggatgcatttcagaagtgtgtatcttctttgagaagcctcaccataatcggaaatcacggaataaataaagtgatgagactgtctggaagaacagggttggagcatttcactctgttggactcactcgaactttcaaagatagaagaccaggaagatgagggcgaagacaacatcatattctggaaatcctttcctcaaaaccttcgcagtttgagaattgaagactctgacaaaatgacaagtttgcccatggggatgcagtacttaacctccctccaaaccctctatctacaccattgttatgaattgaattcccttccagaatggataagcagcttatcatctcttcaatccctgttcatatacaattgtccagccctaaaatcactaccagaagcaatgcggaacctcacctcccttcagagccttgtgatacggcggtgtccagacctaattgaaagatgcgaagaacccaacggcgaggactatcccaaaattcgacacatctccagaattgaaatatggtag

SEQ ID NO: 8 - protein, 974 aa, Spinacia oleracea
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFQLEELLMLGQGGSKVVVTARSEKTANVIGKRHLYTLECLSPDYSWSLFEMSAFQKGQEQENHHELVRIGKKIVEKCYNNPLVITVVGSLLYGEEISKWQSFERSELAKIGNGDNKILSILKLSYYNLANSLKSCFSYCAVFPKDHEIKKEMLIELWMAQGYVVPLDGGQSIEDAAEEHFVILLRRCFFQDVVKDVYGDVNSVKIHDLMHDVAQEVGREEICIVNDNTKKLADKIRHVGCVVDRDPEIVFLCSNKIRSYIGGNCEKRWVDTLIDNWMCLRVLDLSRSDVKNLPNSIGKLLHLRYLNLSHNGDLLILPDAITRLHNLQTLLLHRCSSLKELPKDFCKLVKLRHLDLSGCEDLIGMPLGMDRLTSLRVLPFFVVGRKEQSVDDELKALKGLTEIKGDIRIRICSNYRIVEGMNDTGGAGYLKSMKHLTGVNIIFDYKGVFVNPGAVLATLEPPSNIKRLEVWHYDGTTIPVWERAEINWAISLSHLTITDSKEGEGEWEVGDAFQKCVSSLRSLTIIGNHGINKVMRLSGRTGLEHFTLLDSLELSKIEDQEDEGEDNIIFWKSFPQNLRSLRIEDSDKMTSLPMGMQYLTSLQTLYLHHCYELNSLPEWISSLSSLQSLFIYNCPALKSLPEAMRNLTSLQSLVIRRCPDLIERCEEPNGEDYPKIRHISRIEIW